(19) United States
(12) Patent Application Publication
(10) Pub. No.: US 2007/0092933 A1
(43) Pub. Date: Apr. 26, 2007

(54) Production of multimeric fusion proteins using a c4bp scaffold

(75) Inventors: Laurence Garnier, Saint-Beauzire (FR); Fergal Hill, Saint-Beauzire (FR); Michel Jullen, Saint-Beauzire (FR)

Correspondence Address: NIXON & VANDERHYE, PC, 901 NORTH GLEBE ROAD, 11TH FLOOR, ARLINGTON, VA, 22203, US

(73) Assignee: AVIDIS SA, Saint-Beauzire (FR)

(21) Appl. No.: 10/523,639

(22) PCT Filed: Aug. 12, 2003
(86) PCT No.: PCT/EP03/08928

§ 371 Date: Oct. 25, 2006

(30) Foreign Application Priority Data
Aug. 14, 2002 (EP) 02292043.3

(51) Int. Cl. C12P 21/06; C07H 21/04; C12N 15/74; C07K 14/705; C12N 1/21

(52) U.S. Cl. 435/69.1; 530/350; 435/252.33; 435/488; 536/23.5

(57) ABSTRACT

The present invention provides a method for obtaining a recombinant fusion protein comprising a scaffold of a C-terminal core protein of C4bp alpha chain, said recombinant fusion protein being capable of forming multimers in soluble form in a prokaryotic host cell, the method including the steps of (i) providing a prokaryotic host cell carrying a nucleicacid encoding said recombinant protein operably linked to a promoter functional in said prokaryotic cell; (ii) culturing the host cell under conditions wherein said recombinant protein is expressed; and (iii) recovering the recombinant protein wherein said protein is recovered in multimeric form without performing a scaffold refolding step.

### INTRODUCTION

This invention relates to methods for producing high yields of fusion proteins and polypeptides comprising a C4bp domain in prokaryotic cells.

### BACKGROUND OF THE INVENTION

The advent of recombinant DNA technology has provided the possibility of large scale production of biologically active proteins for therapeutic use. There are now many recombinant DNA produced products in the clinic or under development, including large proteins such as erythropoietin, small peptides, and antibody fragments.

It is known in the art that a difficulty with proteins is one of half life. Many proteins and peptides have a short half-life in vivo, reducing their usefulness. It has been found that multimerisation of protein and peptide molecules is a way of increasing the half-life of these molecules thus allowing them to exert their activity over a longer time scale. Many functional biological molecules have been found to be more potent in vivo when in the form of an oligomeric structure. This is due to factors such as binding with avidity rather than affinity, and/or the ability to cross-link molecules (e.g. identical receptor subunits as in the insulin receptor that are activated through dimerisation, or non-identical molecules to form signalling complexes on the cell surface, such as in lymphocytes). These properties of increased half-life and avidity enable lower doses of the protein and peptide molecules to be used, thereby reducing costs and dose-dependent side-effects.

Different approaches have been proposed for making multimers of recombinant proteins. For example, chemical linkage of proteins to polymers such as polyethylene glycol has been attempted (Katre et al., (1987) Proc. Natl. Acad. Sci. USA 84, 1487). This technique, however, is cumbersome and requires large amounts of purified material. In antibody molecules, modifications of the disulphide-forming possibilities in the hinge, and other regions of the molecules have been attempted in order to modulate the extent to which antibodies will associate with each other. Results however have been inconsistent and unpredictable. Similarly, use of protein A fusions to generate multimeric antibodies may successfully link antibody fragments, but is of limited application in other fields.

A new multimerisation system using the complement 4 binding protein (C4bp) is described in WO 91/11461. Human C4-binding protein (C4bp) is a plasma glycoprotein of high molecular mass (570 kDa) which has a spider like structure made of seven identical alpha-chains and a single beta-chain. The C4bp alpha chain has a C-terminal core region responsible for assembly of the molecule into a multimer. According to the standard model, the cysteine at position +498 of one C4bp monomer forms a disulphide bond with the cysteine at position +510 of another monomer. A minor form comprising only seven alpha-chains has also been found in human plasma. The natural function of this plasma glycoprotein is to inhibit the classical pathway of complement activation.

WO 91/11461 proposes that the ability of the C4bp protein to multimerise can be used to make fusion proteins comprising all or part of C4bp and a biological protein of interest. The fusion protein will form multimers which provides a platform for the protein of interest, in which said protein has an enhanced serum half-life and increased affinity or avidity for its targets. Fusion proteins of C4bp were targeted as the focus of novel delivery and carrier systems for therapeutic products in WO 91/11461.

Most of the alpha-chain of C4bp is composed of eight tandemly arranged domains of approximately 60 amino acids in length known as complement control protein (CCP) repeats. Inclusion of one or more of these domains was preferred in the fusion proteins described in WO 91/11461, but it has since been demonstrated that all CCPs can be deleted (leaving only the C-terminal 57 amino acids) without preventing multimerisation (Libyh M. T. et al., (1997) Blood 90, 3978). This C-terminal region of C4bp is referred to as the C4bp core.

Libyh et al. (1997), describe a protein multimerisation system which is based on the C-terminal part of the alpha chain of C4bp. The C-terminal part of the C4bp lacks biological function, but is responsible for polymerisation of C4bp in the cytoplasm of CHO cells producing C4bp. Libyh et al. were able to induce spontaneous multimerisation of associated antibody fragments to create homomultimers of scFv fragments using the C4bp fragment. The C-terminal portion of C4bp used was placed C-terminal to the scFv sequence, optionally spaced by a MYC tag.

Oudin et al. (2000, Journal of Immunology, 164, 1505) further use the C4bp core multimerising system for forming hetero-multimeric multi CR1/scFv anti-Rh(D) molecules. The chimera proteins were expressed in a CHO cell line by co-transfection of these cells and by two different vectors (one encoding CR1 and the other encoding ScFv anti Rh-D) and were found to spontaneously multimerise in the cytoplasm of the transfected cells from which they were secreted.

Christiansen et al. (2000, Journal of Virology, 74, 4672) further demonstrate the production of homo-multimeric fusion proteins encompassing the CD46 ectodomain linked to the C4bp core in 293 EBNA cells.

Self-assembling multimeric soluble CD4-C4bp fusion protein have also been demonstrated in Shinya et al. (1999, Biomed & Pharmacother, 53, 471) where the fusion proteins were expressed in 293 cells.

Shinya et al. further suggest that the pharmacokinetic properties of fusion proteins containing the C4bp core domain are modified due to the increase in the in vivo plasma half-life of these recombinant fusion proteins in mice. As the core domain used is of human origin, adverse immunological consequences from its administration to humans would be minimised.

To date, fusion proteins based on C4bp core protein have been expressed in eukaryotic cells. The yields of fusion protein from eukaryotic cells has rarely reached 2 micrograms per millilitre of culture supernatant (Oudin et al. ibid) and this could be achieved only after rounds of gene amplification. This level is too low for the economic production of large quantities of many fusion protein for therapeutic use.

One possible way of achieving higher yields would be to use a prokaryotic expression system. WO91/00567 suggests that prokaryotic host cells may be used in the production of C4bp-based proteins, though there is no experimental demonstration of any such production. A number of considerations however, would suggest that the use of prokaryotic systems would be disadvantageous. In particular, many eukaryotic proteins lose some or all of their active folded structure when expressed in cells such as Escherichia coli. Other eukaryotic proteins denature or are completely inactive when expressed in prokaryotic cells.

C4bp is a secreted protein in mammals, and these are known in the art to be particularly difficult to produce in a correctly folded form in prokaryotes. Proteins with disulphide bridges are particularly problematic, as are those that require oligomerisation. Disulphide bonds are not normally produced in the reducing environment of the bacterial cytoplasm, and when they can form, they can stabilise misfolded or aggregated forms of the protein.

Usually, recombinant proteins expressed in prokaryotes are aggregated inside inclusion bodies within the host prokaryotic cell. These are discrete particles or globules separate from the rest of the cell which contain the expressed proteins usually in an agglomerated or inactive form. The presence of the expressed protein in the inclusion bodies makes it difficult to recover the protein in active soluble form as the necessary refolding techniques are techniques are inefficient and costly. Proteins purified from inclusion bodies have to be laboriously manipulated, denatured and refolded to obtain active functional proteins at relatively poor yields.

With regard to expressing C4bp core fusion proteins in prokaryotic cells, other considerations have also to be taken into account. Firstly, each core monomer retains two cysteine residues, and according to the model of C4bp multimers accepted in the art, these cysteines are required to form inter-molecular disulphide bonds during the assembly of multimers. The reducing environment of the prokaryotic cytosol such as the bacterial cytosol would be expected to prevent the formation of C4bp core multimers by reducing these disulphide bonds.

Secondly, multimers are assembled during passage through the eukaryotic secretion apparatus, which is known to assist protein folding in ways not available in prokaryotes (e.g. the presence of protein disulphide isomerase and unique chaperones). Thirdly, even under conditions where relatively small yields were obtained in eukaryotic cells (micrograms per millilitre), this secretory pathway is unable to produce homogenous protein.

### SUMMARY OF THE INVENTION

The inventors have surprisingly found that fusion proteins of C4bp core are not only efficiently synthesized in prokaryotic cells but that the C4bp core itself is capable of folding correctly, and assembling into homogeneous multimers in the reducing environment of the prokaryotic cytosol. The multimers of C4bp core which are produced in prokaryotic cells surprisingly have been found to contain disulphide bonds.

Further, the inventors have also found that proteins fused to the C4bp core produced in the prokaryotic expression systems retain their functional activity. The present invention therefore provides a method for obtaining a recombinant fusion protein comprising a scaffold of a C-terminal core protein of C4bp alpha chain, said recombinant fusion protein being capable of forming multimers in soluble form in the cytosol of a prokaryotic host cell, the method including the steps of
(i) providing a prokaryotic host cell carrying a nucleic acid encoding said recombinant protein operably linked to a promoter functional in said prokaryotic cell;
(ii) culturing the host cell under conditions wherein said recombinant protein is expressed; and
(iii) recovering the recombinant protein wherein said protein is recovered in multimeric form without performing a scaffold refolding step.

We have found that the yield of protein in cell cultures of the invention can be relatively high, for example greater than 2 mg/l of culture, such as greater than 5 mg/l of culture, preferably greater than 10 mg/l of culture, such as greater than 20 mg/l culture, and even more preferably greater than 100 mg/l culture.

C4bp core fusion proteins of the invention comprise a C4bp core protein sequence fused, at the N- or C-terminus, to a biologically active sequence of interest.

### DESCRIPTION OF THE DRAWINGS

FIG. 1 shows an alignment of C4bp sequences from different species.

FIG. 2 shows purification of the fusion protein db-C4bp (where db is a peptide described in Example 1) from an ion-exchange column.

FIG. 3 shows further purification of db-C4bp on a second ion-exchange column.

FIG. 4 shows purification of db-C4bp on a gel chromatography column.

FIG. 5 shows purification of db-C4bp on an ion-exchange column following a heating step.

FIG. 6 shows further purification of db-C4bp on a gel chromatography column.

FIG. 7 shows the activity of DsbA-C4bp in an insulin assay.

FIG. 8 shows the sequence of the promoter and C4bp coding region in pAVD77.

FIG. 9 shows analysis of C4bp fusion proteins under non-reducing conditions.

### DETAILED DESCRIPTION OF THE INVENTION

Core protein of C4bp alpha chain.

This is referred to herein as the “C4bp core protein”or “core protein”, or “C4bp scaffold”. The terms are used interchangeably. This protein may be a mammalian C4bp core protein or a fragment thereof capable of forming multimers, or a synthetic variant thereof capable of forming multimers.

The sequences of a number of mammalian C4bp proteins are available in the art. These include human C4bp core protein (SEQ ID NO:1). There are a number of homologues of human C4bp core protein available in the art. There are two types of homologue: orthologues and paralogues. Orthologues are defined as homologous genes in different organisms, i.e. the genes share a common ancestor coincident with the speciation event that generated them. Paralogues are defined as homologous genes in the same organism derived from a gene, chromosome or genome duplication, i.e. the common ancestor of the genes occurred since the last speciation event.

For example, a search of GenBank indicates mammalian C4bp core homologue proteins in species including rabbit, rat, mouse and bovine origin (SEQ ID NO:2-5 respectively). Paralogues have been identified in pig (ApoR), guinea pig (AM67) and mouse (ZP3); shown as SEQ ID NO:6-8 respectively.

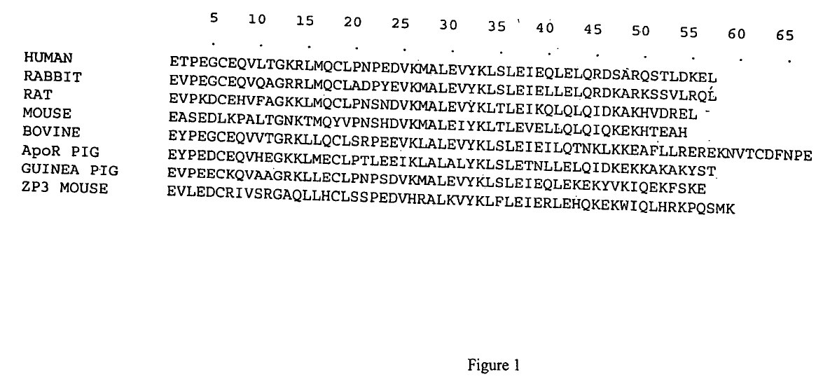

An alignment of SEQ ID NOs:1-8 is shown as FIG. 1. It can be seen that all eight sequences have a high degree of similarity, though with a greater degree of variation at the C-terminal end. Further C4bp core proteins may be identified by searching databases of DNA or protein sequences, using commonly available search programs such as BLAST.

Where a C4bp protein from a desired mammalian source is not available in a database, it may be obtained using routine cloning methodology well established in the art. In essence, such techniques comprise using nucleic acid encoding one of the available C4bp core proteins as a probe to recover and to determine the sequence of the C4bp core proteins from other species of interest. A wide variety of techniques are available for this, for example PCR amplification and cloning of the gene using a suitable source of mRNA (e.g. from an embryo or an actively dividing differentiated or tumour cell), or by methods comprising obtaining a cDNA library from the mammal, e.g. a cDNA library from one of the above-mentioned sources, probing said library with a known C4bp nucleic acid under conditions of medium to high stringency (for example 0.03M sodium chloride and 0.03M sodium citrate at from about 50° C. to about 60° C.), and recovering a cDNA encoding all or part of the C4bp protein of that mammal. Where a partial cDNA is obtained, the full length coding sequence may be determined by primer extension techniques.

A fragment of a C4bp core protein capable of forming multimers may comprise at least 47 amino acids, preferably at least 50 amino acids. The ability of the fragment to form multimers may be tested by expressing the fragment in a prokaryotic host cell according to the invention, and recovering the C4bp fragment under conditions which result in multimerisation of the full 57 amino acid C4bp core, and determining whether the fragment also forms multimers. Desirably a fragment of C4bp core comprises at least residues 6-52 of SEQ ID NO:1 or the corresponding residues of its homologues.

The human C4bp core protein of SEQ ID NO:1 corresponds to amino acids +493 to +549 of full length C4bp protein sequence. A fragment of this known in the art to form multimers corresponds to amino acids +498 to +549 of C4bp core protein.

Variants of C4bp core and fragments capable of forming multimers, which variants likewise retain the ability to form multimers (which may be determined as described above for fragments) may also be used. The variant will preferably have at least 70%, more preferably at least 80%, even more preferably at least 90%, for example at least 95% or most preferably at least 98% sequence identity to a wild type mammalian C4bp core or a multimer-forming fragment thereof.

In one aspect, the C4bp core will be a core which includes the two cysteine residues which appear at positions 6 and 18 of SEQ ID Nos:1-3 and 5-8. Desirably, the variant will retain the relative spacing between these two residues.

The above-specified degree of identity will be to any one of SEQ ID NOs:1-8 or a multimer-forming fragment thereof.

Most preferably the specified degree of identity will be to SEQ ID NO:1 or a multimer-forming fragment thereof.

The degree of sequence identity may be determined by the algorithm GAP, part of the “Wisconsin package” of algorithms widely used in the art and available from Accelrys (formerly Genetics Computer Group, Madison, Wis.). GAP uses the Needleman and Wunsch algorithm to align two complete sequences in a way that maximises the number of matches and minimises the number of gaps. GAP is useful for alignment of short closely related sequences of similar length, and thus is suitable for determining if a sequence meets the identity levels mentioned above. GAP may be used with default parameters.

Synthetic variants of a mammalian C4bp core protein include those with one or more amino acid substitutions, deletions or insertions or additions to the C- or N-termini. Substitutions are particularly envisaged. Substitutions include conservative substitutions. Examples of conservative substitutions include those set out in the following table, where amino acids on the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |
| OTHER | | N Q D E |

Examples of fragments and variants of the C4bp core protein which may be made and tested for their ability to form multimers thus include SEQ ID NOs: 9 to 16, shown in Table 1 below:

| | | |
|---|---|---|
| A | B | C |

| | | |
|---|---|---|
| 9 | -----CEQVLTGKRLMQCLPNPEDVKMALEVYKLSLEIEQLE | 100 |
| | LQRDSARQSTLDKEL | |
| 10 | ETPEGCEQVLTGKRLMQCLPNPEDVKMALEVYKLSLEIKQLE | 98 |
| | LQRDSARQSTLDKEL | |
| 11 | -----CEQVLTGKRLMQCLPNPEDVKMALEVYKLSLEIKQLE | 98 |
| | LQRDSARQSTLDKEL | |
| 12 | ETPEGCEQVLTGKRLMQCLPNPEDVKMALEIYKLSLEIEQLE | 98 |
| | LQRDSARQSTLDKEL | |
| 13 | ETPEGCEQVLTGKRLMQCLPNPEDVKMALEIYKLSLEIKQLE | 96.5 |
| | LQRDSARQSTLDKEL | |
| 14 | ---EGCEQALTGKRLMQCLPNPEDVKMALEIYKLSLEIKQLE | 94 |
| | LQRDSARQSTL---- | |
| 15 | ETPEGSEQVLTGKRLMQSLPNPEDVKMALEVYKLSLEIKQLE | 94 |
| | LQRDSARQSTLDKEL | |
| 16 | ---EGSEQALTGKRLMQSLPNPEDVKMALEIYKLSLEIEQLE | 92.3 |
| | LQRDSARQSTLDK-- | |

A=SEQ ID NO:; B=sequence, C=% identity, calculated by reference to a fragment of SEQ ID NO:l of the same length.

Where deletions of the sequence are made, apart from N- or C-terminal truncations, these will preferably be limited to no more than one, two or three deletions which may be contiguous or non-contiguous.

Where insertions are made, or N- or C-terminal extensions to the core protein sequence, these will also be desirably limited in number so that the size of the core protein does not exceed the length of the wild type sequence by more than 20, preferably by more than 15, more preferably no more than 10, amino acids. Thus in the case of SEQ ID NO:1, the core protein, when modified by insertion or elongation, will desirably be no more than 77 amino acids in length.

N- or C-terminal extensions may include flexible linkers such as (Gly-Gly-Gly-Gly-Ser)ₙ (where n is from 1 to 4) used in the art to attach protein domains (particularly antibody V domains) to each other.

When the fusion proteins of the invention are made by chemical synthesis, N- or C-terminal extensions may include analogues of amino acids not naturally present in proteins which can be used in the art of peptide and polypeptide synthesis.

Recombinant Protein.

The recombinant protein of the invention will comprise a C4bp core (or “scaffold”) as described above either alone or linked in-frame to at least one sequence of biological interest. Such a sequence may comprise a tag useful for identification or purification of the protein, and/or a protein useful in therapy, particularly human therapy.

The recombinant protein can be described as having a general structure of the formula: Bi_{N}-Co-Bi_{C} in which Co is the core protein as described above, and Bi_{N} is either the amino terminus of the core protein or at least one sequence (for example one or two) of biological interest, and Bi_{C} is either the C-terminus of the core protein or at least one sequence (for example one or two) of biological interest.

Preferably, one of Bi_{N} and Bi_{C} is not a sequence of biological interest (i.e. one or other is a terminal of the fusion or optionally a tag, such as a polyhistidine tag, to aid recovery of the protein). More preferably, the biological sequence of interest is represented by Bi_{N}.

Alternatively, a protein or non-protein product of interest may be coupled by synthetic means to a side-chain of the core, e.g. through the amino group of the side-chain of a lysine residue or through cysteine residues added within, or at the end of, the core sequence; or to the existing cysteine residues.

It is preferred that the biological sequence of interest is not all or part of a C4 binding protein normally linked to the C4bp core protein, i.e., the biological sequence of interest is a heterologous sequence.

We have found that proteins falling within the above definition can be expressed in and recovered from bacterial expression systems in multimeric form without the need for scaffold refolding. We have expressed proteins which have a monomer weight up to about 30 kDa. The invention may thus be used to express proteins in this size range, and more generally for proteins up to about 100 kDa, more preferably about 50 kDa.

A particular class of fusion proteins will be those in which the C4bp core is fused to a peptide of from 2 to 25 amino acid residues. Many biologically active peptides are known or can be selected through phage display. However, they are often unstable in vivo, not least because they can be filtered through the renal glomerulus. Fusing them to the core scaffold makes filtration impossible. In addition, it confers avidity on the oligomerised peptides (such that they bind their targets more tightly and are effective at lower doses, and can cross-link receptors). Particular biologically active peptides of interest include naturally occurring peptide or polypeptide hormones, such as somatostatin, calcitonin and alpha-MSH (melanocyte stimulating hormone) and variants thereof as well as other mentioned elsewhere herein.

Thus a range of fusion proteins of C4bp core protein may synthesized using the method of the present invention. The multimeric fusion proteins produced will be expected to exhibit increased bioactivity because multimers will have a higher density of the moiety attached to the C4bp core protein and would thus be expected to have a longer half life and an decreased turnover rate.

The sequence(s) of biological interest may be a polypeptide or a chemical compound (e.g. a drug or pro-drug) or a carbohydrate which is heterologous to the C4bp core protein used in the invention. In other words, it is not part of the same molecule in nature. It may be derived from the same organism. When the attached moiety is a chemical compound, the attachment may serve to protect the compound from metabolism and excretion, for example by hepatic cytochromes, as well as serving to deliver it to tissues. Examples of polypeptides include those used for medical or bio-technological use, such as insulin, cytokines including interleukins and interferons, antibodies and their fragments, growth factors, receptors, receptor ligands, agonists or antagonists, enzymes, enzyme antagonists, antigens, toxins and proteases.

Fusion proteins prepared according to the invention, and the novel fusion proteins of the invention described herein, may be prepared in the form of a pharmaceutical composition which comprises the protein together with one or more pharmaceutically acceptable carriers or diluents. The composition will be prepared according to the intended use and route of administration of the fusion protein.

Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. The active compound as defined above may be formulated as suppositories using, for example, polyalkylene glycols, acetylated triglycerides and the like, as the carrier. Liquid pharmaceutically administrable compositions can, for example, be prepared by dissolving, dispersing, etc, a fusion protein of the invention optional pharmaceutical adjuvants in a carrier, such as, for example, water, saline aqueous dextrose, glycerol, ethanol, and the like, to thereby form a solution or suspension. If desired, the composition to be administered may also auxiliary substances such as pH buffering agents and the like. Actual methods of preparing such dosage forms are known, or will be apparent, to those skilled in this art; for example, see Remington's Pharmaceutical Sciences, Mack Publishing Company, Easton, Pa., 19th Edition, 1995.

The composition or formulation to be administered will, in any event, contain a quantity of the active compound(s) in an amount effective to alleviate the symptoms of the subject being treated. Dosage forms or compositions containing active ingredient in the range of 0.25 to 95% with the balance made up from non-toxic carrier may be prepared.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. A more recently devised approach for parenteral administration employs the implantation of a slow-release or sustained-release system, such that a constant level of dosage is maintained. See, e.g., U.S. Pat. No. 3,710,795.

The following classes of polypeptides are preferred, but the invention is not limited thereto:

Cytokines

Interleukins include any known interleukin including IL-1, IL-2, IL-3, IL-3, IL-4, IL-5, IL-6, IL-7, IL-8, IL-9, IL-10, IL-11 and IL-12. Interleukins are modulators of the immune system. Some interleukins are involved in the inflammatory response or in the immune response to disease.

Interferons include any form of IFN-alpha, as well as IFN-beta and IFN-gamma. These also have use in modulation of the immune response.

A further class of cytokines are the tumour necrosis factors TNF-alpha and TNF-beta.

Other cytokines include members of the MIP family including MIP-1α, MIP-1β and RANTES. RANTES binds the CCR5 HIV co-receptor and therapy with RANTES may be effective in alleviating the progression of HIV infection.

Antibodies

The affinity of antibodies or antibody fragments for antigens may be increased by oligomerisation when the antibodies are produced as C4bp core fusion proteins according to the method of the present invention. Antibody fragments may be fragments such as Fv, Fab and F(ab′)₂ fragments or any derivatives thereof, such as a single chain Fv fragments. The antibodies or antibody fragments may be non-recombinant, recombinant or humanised. The antibody may be of any immunoglobulin isotype, e.g., IgG, IgM, and so forth.

In another aspect, the antibody fragments may be camelised V_{H} domains. It is known that the main intermolecular interactions between antibodies and their cognate antigens are mediated through V_{H} CDR3. However, V_{H}-only antibodies, such as those derived from camel or llama (naturally V_{H}-only single chain antibodies), have only low affinity for cognate antigen.

The method of the present invention makes it possible to obtain improved yields of oligomers of C4bp core proteins with V_{H} domains, or V_{H} CDR3 domains which are high-affinity antibodies. Two or more domains may be included to the C4bp core oligomer made according to the method of the present invention; up to 8 domains may be included, forming an octameric antibody molecule.

Antibody targets may include tumour-associated antigens, including CEA and erbB, which are found in many colon and breast tumours respectively.

In one embodiment, the biological protein of interest may comprise the antibody fused to an enzyme capable of converting a prodrug into a drug toxic to the tumour cell. This can be used in a method of antibody-directed enzyme-prodrug therapy (ADEPT). Alternatively, monomers of carrying a tumour directed antibody and monomers carrying such an enzyme (e.g. a carboxypeptidase, a nitroreductase or the like) may be co-expressed in a cell or expressed in separate cells and mixed together to form heteromultimers directed to a tumour cell.

Antibodies may also be targeted to antigens of pathogenic organisms, including those mentioned below in the context of antigens for use as immunogens.

Growth Factors

Growth factors include hormones such as growth hormone (in particular human growth hormone, hGH, as well as monocyte colony stimulating factor (M-CSF), granulocyte colony stimulating factor (G-CSF), granulocyte macrophage colony stimulating factor (GM-CSF), erythropoietin and platelet derived growth factor (PDGF). Active fragments of such growth factors may also be used. Mammalian, particularly human, growth factors are particularly preferred.

Receptors

Receptors may be useful therapeutically in binding to proteins in the human body which are expressed at aberrant or unwanted levels.

For example, over-expression of TNF-alpha is associated with rheumatoid arthritis, and anti-TNF therapy has been successful in treatment of this condition. The biological protein of interest may thus be a TNF-alpha receptor.

A receptor of interest is also another member of the TNF receptor family, known as the BAFF receptor (Thompson et al. Science, 2001, 293, 2108). The human BAFF receptor (Genbank Accession no. AF373846) is a 184 amino acid protein which binds the TNF-related ligand BAFF. Over-expression of this ligand in mice can cause a systemic lupus erythematosis (SLE)-like symptom, and thus the BAFF receptor is of interest as a possible therapeutic of this disease.

In one aspect, the invention provides a fusion protein of the C4bp core and a BAFF receptor, including fragment of the extracellular domain thereof capable of binding a BAFF ligand. Such a fragment may correspond to amino acids 2-51 of BAFF.

Cell surface receptors are also of interest. For example, CD4 receptor is a target for the HIV surface protein gp120/160, and it has been widely proposed in the art to use CD4, or a soluble fragment thereof, as a therapeutic for HIV infection such that the CD4 blocks the ability of circulating HIV to enter CD4+ T-cells.

Other cell surface receptors are also associated with viral infection, for example CD46 with measles virus (Christiansen et al, ibid), and such cell surface receptor proteins may also be used in the present invention.

Receptor Ligands, Agonists or Antagonists

Many cell surface receptors are activated by dimerisation. Well known examples are those for insulin and erythropoietin. The function of the ligand is to bind simultaneously to two receptors, thus dimerising and activating them. In the examples cited, receptor autophosphorylation occurs. This activates the receptor, which has a tyrosine kinase domain in its intracellular portion. The kinase is inactive when the receptor is monomeric, but is activated on dimerisation. This triggers a cascade of intracellular events, collectively referred to as signal transduction.

Whilst some ligands, such as substance P, are short polypeptides, others (including insulin (51 amino acids) as well as kinase and phosphatase substrates) are complex molecules which possess binding loops projecting from the surface thereof. Smaller molecules which can mimic the natural ligands for receptors are useful for research purposes (for example to understand the specificity of ligand receptor binding).

Short peptides or loops may be incorporated into fusion proteins according to the present invention to form a polyvalent receptor ligand or kinase/phosphatase substrate, useful for activating or inhibiting receptors and/or kinases at very low concentrations.

Variation may be introduced into the heterologous polypeptides inserted onto the scaffold in order to map the specificity of receptors or kinases/phosphatases for their ligands or substrates. Variants may be produced of the same loop, or a set of standard different loops may be devised, in order to assess rapidly the specificity of a novel kinase/phosphatase.

Variants may be produced by randomisation of sequences according to known techniques, such as PCR. They may be subjected to selection by a screening protocol, such as phage display, before incorporation into protein scaffolds in accordance with the invention.

Agonists include peptides, including peptide mimetics, which bind to a receptor so as to trigger the action of the receptor in even in the absence of the natural ligand for that receptor. An example of an agonist is the thrombopoeitin agonist peptide. This linear 14-mer peptide is found to be 4,000-fold more active when dimeric than when monomeric (Dower W. J. et al. Stem Cells (1998) 16, Suppl 2, 21 Peptide agonists of the thrombopoietin receptor). Fusion of this sequence, IEGPTLRQWLAARA, to the core domain of C4bp, as described below for other peptides should create a very potent thrombopoietin agonist, useful for promoting platelet production and/or maturation.

In a further aspect, the invention provides a recombinant protein comprising a C4bp core protein and a thrombopoeitin agonist peptide, and the use of such a protein in a method of therapy for promoting platelet production and/or maturation in a human subject. The method comprises administering to a subject in need of treatment an effective amount of the protein.

A further example of an agonist is the somatostatin peptide. This cyclic peptide is known to bind to a number of G-protein coupled receptors, and to inhibit the release of somatotropin. An analogue is marketed as Sandostatin (Novartis) for a number of medical indications, including the treatment of side effects associated with malignant carcinoid tumours and the treatment of diarrhea caused by gastrointestinal infections.

Fusion of the somatostatin sequence to filamentous bacteriophage, as described in the British Journal of Pharmacology (1998) “Somatostatin displayed on filamentous phage as a receptor-specific agonist”. Volume 125, pages 5-16, produces a hybrid phage capable of binding to and activating somatostatin receptors. Fusion of somatostatin to the C4bp scaffold (with the scaffold replacing the phage) similarly produces an avid agonist for somatostatin receptors, which has more desirable properties as a medicament than hybrid phage. Similarly, the oligomeric agonist so produced is capable of oligomerising the somatostatin receptors, which may enhance signalling, as described by Patel et al. in Proc. Natl. Acad. Sci. USA (2002) Volume 99, pages 3294-3299.

Thus the invention provides a means to prepare a recombinant fusion protein as set out above wherein said fusion protein comprises somatostatin. The invention further provides a fusion protein of a C-terminal core protein of C4bp alpha chain linked to somatostatin. The invention further provides the use of this protein or nucleic acid vectors (as further defined and described herein) encoding this protein in a method of treatment, including the treatment of side effects associated with malignant carcinoid tumours and the treatment of diarrhea caused by gastrointestinal infections.

Antagonists include peptides which bind to receptors and block the natural ligand from binding.

Enzymes

Numerous biological reactions involve the sequential, and/or synergistic, action of a plurality of protein activities. Such protein activities may be incorporated into a single molecule in accordance with the present invention. Preferably, therefore, the monomers which are used to compose the oligomer according to the invention incorporate amino acid sequences which encode distinct biological activities. The activities are advantageously complementary, such that they are required sequentially in a biological reaction, or act synergistically. The invention therefore provides plurifunctional macromolecular structures comprising one or more enzymes.

Examples of enzymes include bacterial enzymes such as DsbA of E. coli.

Antigens

A particular use for multimers of produced in accordance with the invention is in the production of immunogens (this term is used interchangeably herein with “antigens”). A major application of this C4bp core fusion protein scaffold technology produced following the method of the present invention is the use of the assembled or multimerised peptides or polypeptides as antigens. The oligomerisation improves both detection of antibodies against, and the induction of antibodies to, such antigens. Some of these antigens may be of prophylactic value; they might be useful for vaccination. The method allows rapid progress from nucleotide sequences to the production of recombinant antigens in a polyvalent form. Predicted open reading frames (ORFs) can be used to design oligonucleotide sequences encoding the predicted protein sequence. Cloning of these oligonucleotides into the vectors encoding the C4bp core protein allows a very rapid production of antigens, without, for example the need for isolating cDNAs and expressing them in heterologous systems such as E. coli.

Bacterial immunogens, parasitic immunogens and viral immunogens are useful as polypeptide moieties to create multimeric or hetero-multimeric C4bp fusion proteins useful as vaccines.

Bacterial sources of these immunogens include those responsible for bacterial pneumonia, pneumocystis pneumonia, meningitis, cholera, tetanus, tuberculosis and leprosy.

Parasitic sources include malarial parasites, such as Plasmodium.

Viral sources include poxviruses, e.g., cowpox virus and orf virus; herpes viruses, e.g., herpes simplex virus type 1 and 2, B-virus, varicellazoster virus, cytomegalovirus, and Epstein-Barr virus; adenoviruses, e.g., mastadenovirus; papovaviruses, e.g., papillomaviruses such as HPV16, and polyomaviruses such as BK and JC virus; parvoviruses, e.g., adeno-associated virus; reoviruses, e.g., reoviruses 1, 2 and 3; orbiviruses, e.g., Colorado tick fever; rotaviruses, e.g., human rotaviruses; alphaviruses, e.g., Eastern encephalitis virus and Venezuelan encephalitis virus; rubiviruses, e.g., rubella; flaviviruses, e.g., yellow fever virus, Dengue fever viruses, Japanese encephalitis virus, Tick-borne encephalitis virus and hepatitis C virus; coronaviruses, e.g., human coronaviruses; paramyxoviruses, e.g., parainfluenza 1, 2, 3 and 4 and mumps; morbilliviruses, e.g., measles virus; pneumovirus, e.g., respiratory syncytial virus; vesiculoviruses, e.g., vesicular stomatitis virus; lyssaviruses, e.g., rabies virus; orthomyxoviruses, e.g., influenza A and B; bunyaviruses e.g., LaCrosse virus; phleboviruses, e.g., Rift Valley fever virus; nairoviruses, e.g., Congo hemorrhagic fever virus; hepadnaviridae, e.g., hepatitis B; arenaviruses, e.g., 1 cm virus, Lasso virus and Junin virus; retroviruses, e.g., HTLV I, HTLV II, HIV-1 and HIV-2; enteroviruses, e.g., polio virus 1,-2 and 3, coxsackie viruses, echoviruses, human enteroviruses, hepatitis A virus, hepatitis E virus, and Norwalk- virus; rhinoviruses e.g., human rhinovirus-; and filoviridae, e.g., Marburg (disease) virus and Ebola virus.

Antigens from these bacterial, viral and parasitic sources may be used in the production of multimeric proteins useful as vaccines. The multimers may comprise a mixture of monomers carrying different antigens.

Immunogens to human proteins for research or therapeutic purposes may be made. Immunogenic peptides, capable of raising an immune response when exposed to the immune system of an organism, are preferred polypeptides for making C4bp core protein fusion proteins following the method of the invention. The improved yield of oligomerised C4bp core fusion proteins from the present invention has many applications not only in vaccination but also in research. For example, the generation of human gene sequence data by the human genome project has made the generation of antisera reactive to new polypeptides a pressing requirement. The same requirement applies to prokaryotic, such as bacterial, and other eukaryotic, including fungal, gene products. Immunogens of interest fused to C4bp core multimers are thought to have increased efficiency due to their increased avidity for immunoglobulin molecules.

The present invention has many advantages in the generation of an immune response. For example, the use of oligomers can permit the presentation of a number of antigens, simultaneously, to the immune system. This allows the preparation of polyvalent vaccines, capable of raising an immune response to more than one epitope, which may be present on a single organism or a number of different organisms. Thus, vaccines formed according to the invention may be used for simultaneous vaccination against more than one disease, or to target simultaneously a plurality of epitopes on a given pathogen. The epitopes may be present in a single monomer units or on different monomer units which are combined to provide a heteromultimer.

Moreover, the invention may be exploited by incorporating an adjuvant on the C4bp core oligomer, together with the immunogen. Suitable adjuvants are, for example, bacterial toxins and cytokines, such as interleukins. The potency of the immunogen is thereby increased, allowing more efficient raising of antisera and more efficient immunisation. A highly preferred adjuvant is the C3d component of complement.

Having C4bp core fusion proteins is useful in the context of immunisations, because the core protein is not only present normally in the serum or plasma of the recipient of the immunisation, but also because it does not itself evoke an immune response. C4bp proteins are known in a number of mammalian species, and the appropriate homologues for mammalian species may be found by those skilled in the art using standard gene cloning techniques.

The fact that this system allows production of soluble protein in E. coli enables using it to produce, as folded soluble proteins, domains or fragments of proteins that would not fold when expressed on their own due to a lack of constraint on their C-terminal and/or N-terminal ends. Engineering a specific cleavage site enables production of the free domain of interest. Similarly constraining the N-terminal and/or C-terminal end of a peptide of interest could be beneficial during refolding processes. Furthermore, as the oligomerisation structure is very resistant to denaturation and to disassembly, it would be stable during denaturation of the inserted protein. Therefore, during refolding, for an equal amount of protein of interest, the actual concentration of free protein would be diminished by a factor equal to the oligomerisation number. Oligomerisation may also be beneficial for purification purposes as many methods in protein technology are not optimised to work with proteins and specifically peptides of very low molecular weight.

Assay Methods

The C4bp core fusion proteins produced following the method of the invention may be applied to the detection or the neutralisation of antibodies in vivo or in vitro. For example, in vitro polyvalent or monovalent antigen-bearing C4bp core fusion proteins may be used to select antibody molecules derived from phage display experiments. Moreover, in vivo, antigen-bearing C4bp core fusion proteins produced according to the method of invention may be used to neutralise antoantibodies in autoimmune disease, or to detect antibodies which may be indicative of pathological conditions, such as in HIV testing or other diagnostic applications.

Phage Display

Phage display technology has proved to be enormously useful in biological research. It enables ligands to be selected from large libraries of molecules. The proteins of the present invention also harnesses the power of this technique, but with some powerful advantages over normal applications. C4bp molecules can be displayed as monomers on fd bacteriophages, just as single-chain Fv molecules are. Libraries of fusions are constructed by standard methods, and the resulting libraries screened for ligands of interest. It is important to note that this is an affinity based selection. After characterisation, the ligands selected for affinity, can be oligomerised, and thus take advantage of avidity. When the target for the ligand is oligomeric, very tight binding will result. Furthermore, ligands selected as monomers, will be able to cross-link or oligomerise their binding partners. An application of this effect is in triggering receptor activation.

Protein Chips

Currently, DNA microarrays, whether of oligonucleotides, PCR products or cloned DNAs, are major tools enabling rapid development in the highly parallel analysis of gene expression. Clearly, in many situations, it would be far preferable to monitor gene expression directly, that is, by assaying protein expression levels rather than mRNA levels. The latter are but an indirect measure of gene activity which rely on the hybridisation of labelled cDNA and can be very misleading because there is often a poor correlation between the abundance of a particular mRNA and the frequency at which it is translated into proteins. In addition, mRNA analysis can not possibly determine whether the encoded protein, even if translated, is active. This may depend on post-translational modification.

Thus protein arrays comprising fusion proteins of a core scaffold and a range of ligands for proteins of interest may be produced and used to determine levels of expression of those proteins in a sample.

For example, an array of bacterial cells expressing the scaffold-ligand fusions may be provided, such that the fusions are expressed and recovered in situ, followed by addition of the sample. Alternatively, the fusions may be produced separately and then arrayed on a suitable solid support to provide for detection of the proteins in the sample. Detection may be by providing a predetermined amount of the proteins of interest labelled to compete against the proteins present in the sample, and measuring how much labelled protein binds to the ligand. Alternatively, the ligand may be labelled and the amount of labelled ligand bound to the protein of interest detected.

Nucleic Acids

Proteins comprising the C4bp core are produced by expression of the protein in a prokaryotic host cell, using a nucleic acid construct encoding the recombinant protein.

The construct will generally be in the form of a replicable vector, in which sequence encoding the protein is operably linked to a promoter suitable for expression of the protein in a desired host cell. The promoter may be an inducible promoter. Suitable promoters include the T7 promoter, the tac promoter, the trp promoter, the lambda promoters P_{L} or P_{R} and others well known to those skilled in the art.

The vectors may be provided with an origin of replication and optionally a regulator of the promoter. The vectors may contain one or more selectable marker genes, for example an antibiotic resistance gene such as an ampicillin, tetracycline or preferably kanamycin resistance gene. There are a wide variety of bacterial expression vectors known as such in the art, and the present invention may utilise any vector according to the individual preferences of those of skill in the art.

A wide variety of prokaryotic host cells can be used in the method of the present invention. These hosts may include strains of Escherichia, Pseudomonas, Bacillus, Lactobacillus, Thermophilus, Salmonella, Enterobacteriacae or Streptomyces. For example, if E. coli from the genera Escherichia is used in the method of the invention, preferred strains of this bacterium to use would include BL21(DE3) and their derivatives including C41(DE3), C43(DE3) or CO214(DE3), or other strains resistant to the toxicity of recombinant protein expression as described and made available in WO98/02559.

Even more preferably, derivatives of these strains lacking the prophage DE3 may be used when the promoter is not the T7 promoter.

DNA Vaccines and Therapeutics

In another aspect, the invention provides a eukaryotic expression vector comprising a nucleic acid sequence encoding a recombinant fusion protein comprising a scaffold of a C-terminal core protein of C4bp alpha chain for the use in the treatment of the human or animal body.

Such treatment would achieve its therapeutic effect by introduction of a specific nucleic acid sequence into cells or tissues affected by a genetic or other disease, or by introduction of a nucleic acid sequence encoding an antigen for the purposes of raising an immune response. It is also possible to introduce genetic sequences into a different cell or tissue than that affected by the disease, with the aim that the gene product will have direct or indirect impact on the diseases cells or tissues. Delivery of nucleic acids can be achieved using a plasmid vector (in “naked” or formulated form) or a recombinant expression vector.

Various viral vectors which can be utilized for gene therapy include adenovirus, herpes virus, vaccinia or an RNA virus such as a retrovirus. The retroviral vector may be a derivative of a murine or avian retrovirus. Examples of retroviral vectors in which a single foreign gene can be inserted include, but are not limited to: Moloney murine leukaemia virus (MoMuLV), Harvey murine sarcoma virus (HaMuSV), murine mammary tumour virus (MuMTV), and Rous Sarcoma Virus (RSV). When the subject is a human, a vector such as the gibbon ape leukaemia virus (GaLV) can be utilized.

The vector will include a transcriptional regulatory sequence, particularly a promoter region sufficient to direct the initiation of RNA synthesis. Suitable eukaryotic promoters include the promoter of the mouse metallothionein I gene (Hamer et al. 1982 J. Molec. Appl. Genet. 1, 273); the TK promoter of Herpes virus (McKnight, 1982 Cell 31,355); the SV40 early promoter (Benoist et al. 1981 Nature 290, 304); the Rous sarcoma virus promoter (Gorman et al. 1982 Proc. Natl Acad. Sci. USA 79, 6777); and the cytomegalovirus promoter (Foecking et al. 1980 Gene 45, 101).

Promoters specific for the cell type requiring the gene therapy are desirable in many instances. In a situation where a particular cell type is used as a platform to produce therapeutic proteins destined for another site (for either direct or indirect action), then the chosen promoter should work well in the “factory” site. Muscle is a good example for this, as it is post-mitotic, it could produce therapeutic proteins for years on end as long as there is no immune response against the protein-expressing muscle fibres. Therefore, use of strong muscle promoters as are particularly applicable here. Except for treating a muscle disease per se, use of muscle is typically only suitable where there is a secreted protein so that it can circulate and function elsewhere (e.g., hormones, growth factors, clotting factors).

Administration of vectors of this aspect of the invention to a subject, either as a plasmid vector or as part of a viral vector can be affected by many different routes. Plasmid DNA can be “naked” or formulated with cationic and neutral lipids (liposomes) or microencapsulated for either direct or indirect delivery. The DNA sequences can also be contained within a viral (e.g., adenoviral, retroviral, herpesvius, pox virus) vector, which can be used for either direct or indirect delivery. Delivery routes include but are not limited to intramuscular, intradermal (Sato, Y. et al. 1996 Science 273, 352), intravenous, intra-arterial, intrathecal, intrahepatic, inhalation, intravaginal instillation (Bagarazzi et al. 1997 J Med. Primatol. 26, 27), intrarectal, intratumour or intraperitoneal.

Thus the invention includes a vector as described herein as a pharmaceutical composition useful for allowing transfection of some cells with the DNA vector such that a therapeutic polypeptide will be expressed and have a therapeutic effect (to ameliorate symptoms attributable to infection or disease). The pharmaceutical compositions according to the invention are prepared by bringing the construct according to the present invention into a form suitable for administration to a subject using solvents, carriers, delivery systems, excipients, and additives or auxiliaries. Frequently used solvents include sterile water and saline (buffered or not). One carrier includes gold particles, which are delivered biolistically (i.e., under gas pressure). Other frequently used carriers or delivery systems include cationic liposomes, cochleates and microcapsules, which may be given as a liquid solution, enclosed within a delivery capsule or incorporated into food.

An alternative formulation for the administration of gene therapy vectors involves liposomes. Liposome encapsulation provides an alternative formulation for the administration of polynucleotides and expression vectors. Liposomes are microscopic vesicles that consist of one or more lipid bilayers surrounding aqueous compartments. See, generally, Bakker-Woudenberg et al. 1993 Eur. J. Clin. Microbiol. Infect. Dis. 12, Suppl. 1, S61, and Kim, 1993 Drugs 46, 618. Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02 μM to greater than 10 μM. See, for example, Machy et al. 1987 LIPOSOMES IN CELL BIOLOGY AND PHARMACOLOGY (John Libbey), and Ostro et al. 1989 American J. Hosp. Phann. 46, 1576.

Expression vectors can be encapsulated within liposomes using standard techniques. A variety of different liposome compositions and methods for synthesis are known to those of skill in the art. See, for example, U.S. Pat. Nos. 4,844,904, 5,000,959, 4,863,740, 5,589,466, 5,580,859, and 4,975,282, all of which are hereby incorporated by reference.

In general, the dosage of administered liposome-encapsulated vectors will vary depending upon such factors as the patient's age, weight, height, sex, general medical condition and previous medical history. Dose ranges for particular formulations can be determined by using a suitable animal model.

In one embodiment, the vector encodes a fusion protein comprising the core and, in addition, one or more antigens and optionally and preferably a protein with immunostimulatory properties. C3d is known to have strong immunostimulatory properties and may be used for this purpose, as may be an interleukin, particularly IL-2 or IL-12.

Cell Culturing

Plasmids encoding fusion proteins in accordance with the invention may be introduced into the host cells using conventional transformation techniques, and the cells cultured under conditions to facilitate the production of the fusion protein. Where an inducible promoter is used, the cells may initially be cultured in the absence of the inducer, which may then be added once the cells are growing at a higher density in order to maximise recovery of protein.

Cell culture conditions are widely known in the art and may be used in accordance with procedures known as such.

Recovery of Protein from Culture

Once the cells have been grown to allow for production of the protein, the protein may be recovered from the cells. Because we have found that surprisingly, the protein remains soluble, the cells will usually be spun down and lysed by sonication which keeps the protein fraction soluble and allows this fraction to remain in the supernatant following a further higher-speed (e.g. 15,000 rpm for 1 hour) centrifugation.

The fusion protein in the supernatant protein fraction may be purified further by any suitable combination of standard protein chromatography techniques. We have used ion-exchange chromatography followed by gel filtration chromatography. Other chromatographic techniques, such as affinity chromatography, may also be used.

In one embodiment, we have found that heating the supernatant sample either after centrifugation of the lysate, or after any of the other purification steps will assist recovery of the protein. The sample may be heated to about 70-80° C. for a period of about 10 to 30 minutes.

Depending on the intended uses of the protein, the protein may be subjected to further purification steps, for example dialysis, or to concentration steps, for example freeze drying.

The invention is illustrated by the following examples.

### EXAMPLE 1

### Production of db-C4bp

Vector Construct.

An expression vector encoding the downstream box peptide sequence MASMNHKGS (Sprengert M. L., Fuchs E. and Porter A. G 1996 “The downstream box: an efficient and independent translation initiation signal in Escherichia coli.” EMBO J. Volume 15, 665-674) fused N-terminal to the 57 amino acid “core” domain of the human C4bp alpha chain was constructed.

Briefly, the C4bp core domain is encoded entirely within a single exon in the human genome, thus allowing it to be amplified directly from human genomic DNA. The oligo-nucleotide primers used were:
AVD102: 5′ CCCGCGGATCCGAGACCCCCGAAGGCTGTGA3′; and
AVD103: 5′ CCCCGGAATTCTTATTATAGTTCTTTATCCAAAGTGG3′.

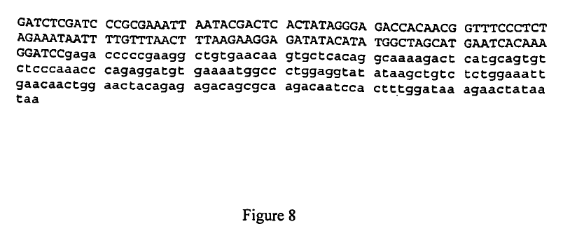

These contained added restriction sites which were used for cloning the amplified DNA fragment. The 183 base-pair fragment obtained on digesting the PCR product with the enzymes BamHI and EcoRI was cloned downstream of the translational enhancer or “downstream box” and the T7 promoter in a plasmid vector. The plasmid was derived from the plasmid pRsetA supplied by Invitrogen, but the f1 origin of replication has been replaced by the par locus from the plasmid pSC101. It thus contains as functional elements: a selectable marker (ampicillin resistance) an origin of replication (derived from the pUC family) and a T7 promoter and a T7 transcription terminator as well as the par locus. The resulting construct was designated plasmid pAVD 77. FIG. 8 shows the sequence of the translational enhancer and T7 promoter fused to the coding sequence of C4bp (in small print).

The predicted size of the db-C4bp fusion protein is 7491.5 Da.

Transformation and Expression.

The vector was transformed into the E. coli strain C41(DE3), a derivative (Bruno Miroux and John E. Walker 1996 “Over-production of Proteins in Escherichia coli: Mutant Hosts that Allow Synthesis of some Membrane Proteins and Globular Proteins at High Levels.” Journal of Molecular Biology Volume 260, 289-298) of BL21(DE3).

One litre of LB-Ampicillin medium was inoculated with the cells, which were incubated at 37° C with shaking for 3 hours (until OD600 nm reached 0.6) and then it was induced with IPTG (isopropylthiogalactoside) at a final concentration 0.7 mM for 3 hours. The cells were harvested by centrifugation at 4600 rpm for 30 min.

The pellet (P) was resuspended with 30 mls Tris 50 mM pH 7, and the cells were broken by sonication using an Emulsiflex apparatus twice (between each treatment, centrifugation at 15000 rpm for 1 hour, the supernatants from each spin (designated SN1 and SN2 respectively) were kept and the pellet P1 was resuspended with the same buffer).

Both supernatants were pooled (60 mls) and were split into two solutions of 30 mls. Each of these 30 ml aliquots of the db-C4bp fusion protein was purified using one of two similar methods: these were identical except that a heating step in one method was replaced by a MonoQ ion-exchange step in the other.

Purification Without a Heating Step

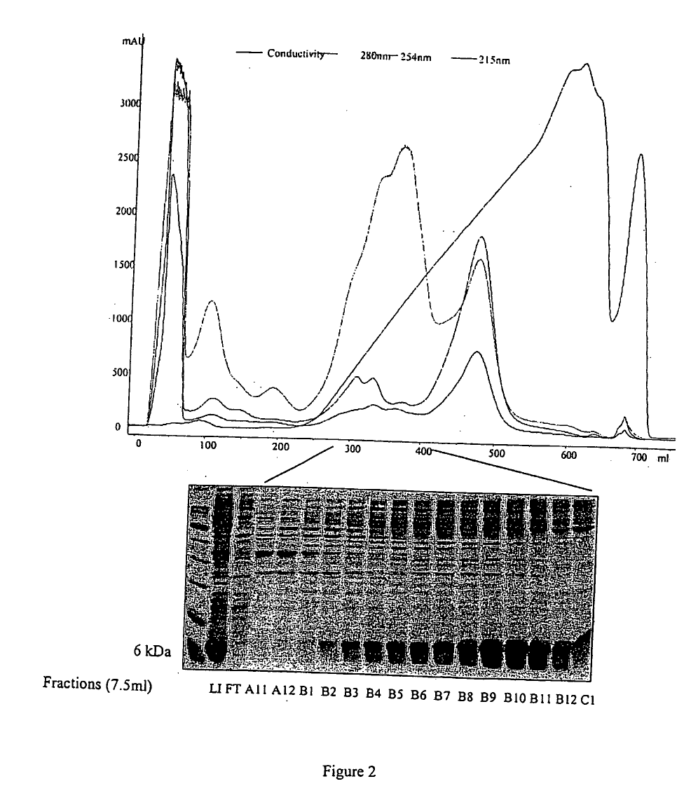

The native db-C4bp was purified from 500 mls of culture by ion-exchange chromatography (DEAE Fast Flow 70, using a column of 13 cm in height, and diameter of 2.6 cm), using TrisHCl buffer (50 mM pH7) and a salt gradient (0M-1M NaCl). The fusion protein eluted between 300-400 mM NaCl. Fractions of 7.5 ml each were collected—see FIG. 2.

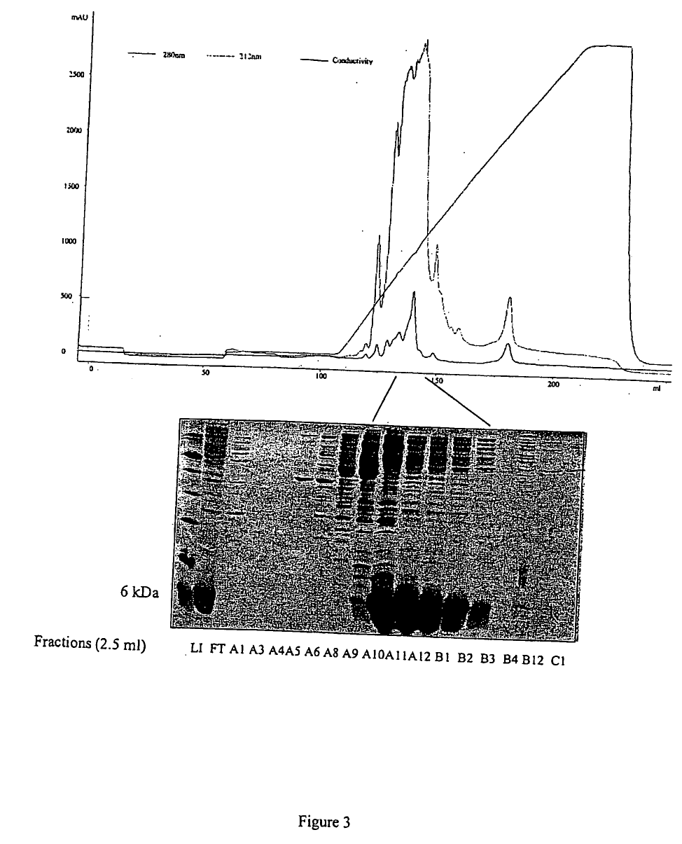

Fractions B8 to B11 were pooled and dialyzed against TrisHCl 20 mM pH7. Then this solution was loaded on a ion-exchange column (MonoQ HR 16/10), using Tris buffer (50 mM pH7) and a salt gradient (0M-1M NaCl). Fractions of 2.5 ml were collected. The fusion protein eluted between 500-550 mM NaCl (FIG. 3).

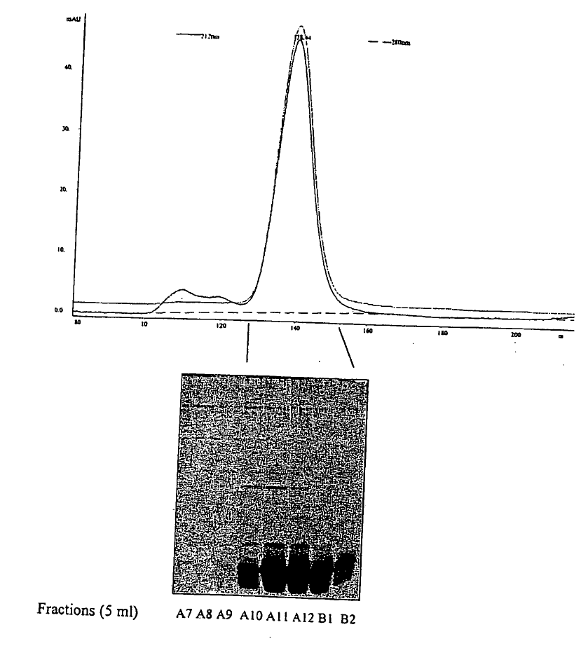

Fractions A10 to B1 were pooled and the final solution was then concentrated to a volume of 10 mls before being chromatographed on a gel filtration column (S75 26/60). Fractions of 5 ml were collected. The fusion protein was eluted from this column with a volume of 139 mls buffer (TrisHCl 100 mM pH7, 150 mM NaCl), see FIG. 4. The calibration of the column with molecular weight standards implies a molecular weight for this protein similar to albumin (67 kDa), which in Tris 50 mM+NaCl 150 mM also elutes with a volume of 139 mls, whereas the expected molecular weight of the monomer is 7.491 kDa. This indicates that the fusion protein is oligomeric in structure when purified from the cytosol of E. coli, without any steps being taken to refold it.

Fractions A10 to B1 were pooled (312 μg/ml), and an aliquot was dialysed against sodium phosphate buffer, 100 mM, pH 7.4.

The protein yield per Litre of culture after purification was 12.4 milligrams.

The CD spectrum was examined and showed the presence of significant secondary structure, consistent with a properly folded protein complex.

### EXAMPLE 2

### Purification of db-C4bp with a Heating Step

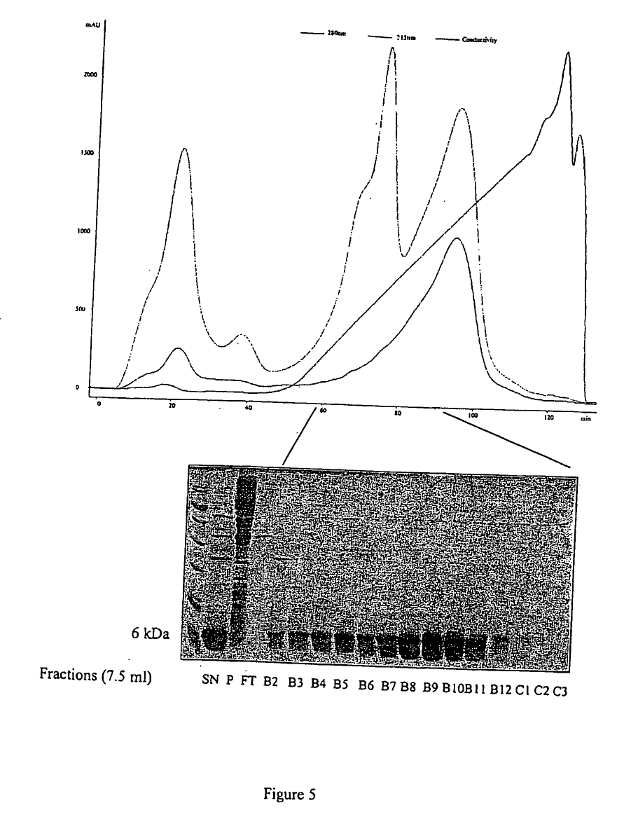

The solution containing the other 30 ml aliquot of db-C4bp was heated at 76° C. for 15 minutes and then centrifuged at 20,500 rpm for 1 hour. The supernatant, containing db-C4bp, was purified by ion-exchange chromatography (DEAE Fast Flow 70 mls), using Tris buffer (5OmM pH7) and a salt gradient (0M-1M NaCl). Fractions of 7.5 ml were collected. The fusion protein eluted between 300-400 mM NaCl (FIG. 5).

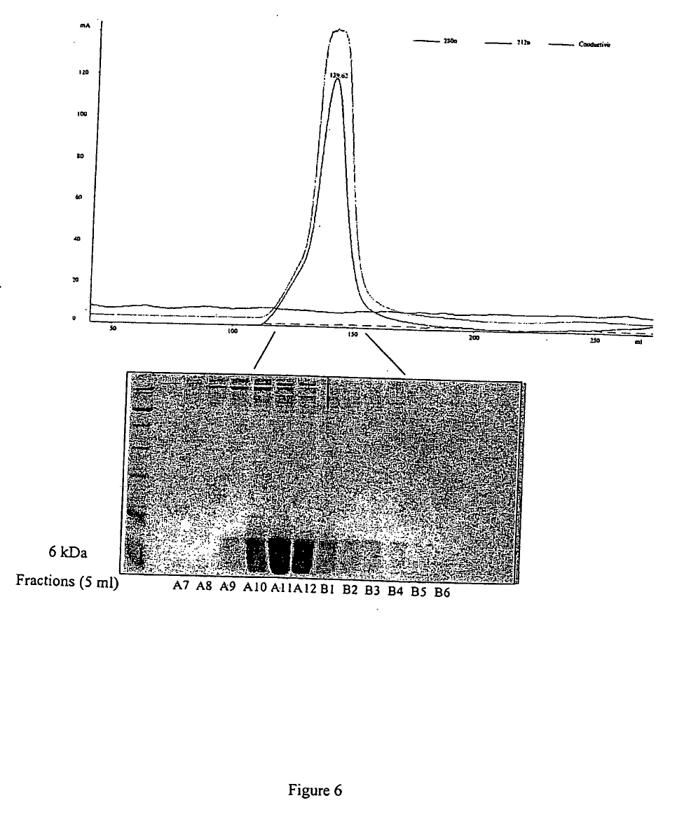

Fractions B8 to B11 were pooled and the final solution was then concentrated to a volume of 10 mls before being chromatographed on a gel filtration column (S-75 26/60). Fractions of 5 ml were collected. The fusion protein was eluted from this column with a volume of 140 mls buffer (FIG. 6). The calibration of the column with molecular weight standards implies a molecular weight identical to that of the protein purified without heating (see above), whereas the expected molecular weight of the monomer is 7.491 kDa. This fusion protein is therefore also oligomeric in structure when purified from the cytosol of E. coli, without any steps being taken to refold it. Furthermore, it remains oligomeric despite being heated to 76° C. for 15 minutes in a buffer comprising 50 mM TrisHCl pH7 (i.e. no salt was present).

Fractions A11 to B1 were pooled (595.5 μg/ml) and an aliquot was dialysed against sodium phosphate (NaP) buffer 100 mM pH 7.4.

Analysis using circular dichroism showed that the spectrum obtained with the sample which had been subjected to heating was equivalent to that obtained using the unheated sample. This demonstrated that the secondary structure elements of the protein are retained despite heating.

The yield with the heating step was 3.5 milligrams per litre.

The addition of a heating step can significantly simplify the purification of proteins. In the example here, heating replaced one ion-exchange (MonoQ) step, and nevertheless resulted in a protein of at least equivalent purity.

### EXAMPLE 3

### Treatment of Protein with Denaturant

To confirm further that the protein was indeed oligomeric, an attempt was made to denature purified protein in 6M guanadinium chloride and 20 mM DTT (dithiothreitol) at room temperature before repeating gel filtration under denaturing conditions.

Briefly, a culture of 500 mls of the cells of example 1 were grown and induced as described above. The fusion protein was purified by ion-exchange chromatography, using TrisHCl buffer (50 mM pH 7.4) and a salt gradient (0M-1M NaCl). The fusion protein eluted between 450-650 mM NaCl and was then concentrated to a volume of 10 mls. After this concentration step, the concentration of db-C4bp protein was 740 micrograms per ml.

The protein was then treated at a concentration of 740 micrograms per ml overnight at 4° C. with 6M guanidinium chloride and 20 mM DTT before being chromatographed on a gel filtration column (S-75). The fusion protein was eluted from this column with a volume of 11.4 mls buffer. Calibration of the column with molecular weight standards implies a molecular weight for this protein of approximately 60 kDa, whereas the expected molecular weight of the monomer is 7.5 kDa. This fusion protein is therefore oligomeric in structure when purified from the cytosol of E. coli, without any steps being taken to refold it and even when treated to denaturing conditions.

Repeating the denaturation step using 6M guanidine HCl for 2 or 16 hours and heating to 75° C.-80° C. did result in denatured protein, as evidenced by CD analysis.

### EXAMPLE 4

### Cloning and Recombinant Expression in E. coli of the Human C4bp Core Fused to a Histidine Tag Sequence

To demonstrate that the translational enhancer is not essential for high-level expression of the core domain in Escherichia coli, and to facilitate the purification of the protein, the DNA sequence encoding the downstream box was replaced by a sequence encoding a 6xHistidine tag by replacing an NdeI/BamHI restriction fragment in pAVD 77 with the following sequence:
CATATGCGGG GTTCTCATCA TCATCATCAT CATGGTCTGG TTCCGCGTGG ATCC

The resulting plasmid pAVD 93, overproduces a recombinant protein of 8.46 kDa with the following amino acid sequence:
MRGSHHHHHH GLVPRGSETP EGCEQVLTGK RLMQCLPNPE DVKMALEVYK LSLEIEQLEL QRDSARQSTL DKEL

The plasmid pAVD 93 was transformed into the bacterial strain C41(DE3) and expression of the fusion protein was induced using IPTG as described in above. A protein of 8.5 kDa as shown by SDS-PAGE analysis was present in induced cultures 3 hours after induction but absent from uninduced cultures.

### EXAMPLE 5

### Cloning and Recombinant Expression in E. coli of the Human C4bp Core Fused to the DsbA Protein

The fusion of the C4bp core domain to the short peptide sequences encoded by the downstream box enhancer or to the histidine tag does not necessarily imply that the fusion of the core domain to larger proteins is feasible. To determine this, the C4bp core was fused to the C-terminus of the DsbA protein, an enzyme normally found in the E. coli periplasmic space. DsbA comprises 177 amino acids, and as such, is substantially larger than the core domain itself (57 amino acids).

Construction of the Plasmid pAVD 78, Encoding the DsbA-C4bp Fusion Protein

The NdeI-BamHI DNA fragment in pAVD 77 encoding the downstream box enhancer was replaced by an NdeI-BamHI fragment encoding DsbA. The oligonucleotide primers used to obtain the fragment encoding DsbA were:
AVD52: 5′ GGGGCCCCCATATGGCGCAGTATGAAGATGGTAAACAG3′; and
AVD115: 5′ GGGGAATTCTTAGGATCCAGAACCTTTTTTCTCGGACAGATATTTCAC3′.

These primers were used to amplify the DsbA coding sequence (lacking a stop codon) from the genomic DNA of Escherichia coli. The PCR product was digested with both NdeI and BamHI restriction enzymes, and cloned into pAVD 77 in to create pAVD 78.

The plasmid pAVD 77 was transformed into the bacterial strain C41(DE3) and expression of the fusion protein was induced using IPTG as described above. A protein of 28 kDa as shown by SDS-PAGE analysis was present in induced cultures 3 hours after induction, but absent from uninduced cultures. Surprisingly, this protein was present in the soluble fraction of the cell extract.

Purification of the DsbA-C4bp Fusion Protein

The fusion protein was purified by two ion-exchange chromatographic steps (first DEAE, secondly MonoQ), using Tris HCl buffer (50 mM pH 7.4) and a salt gradient (0 M-1M NaCl) in each case. The fusion protein eluted after the first (DEAE) ion-exchange chromatography at approximately 100 mM NaCl and was then purified by a more resolutive (MonoQ) ion-exchange chromatography. The fusion protein eluted at 350 mM NaCl from the MonoQ and was concentrated before being chromatographed on a S200 gel filtration column (10/30). The fusion protein was eluted from this column in a volume of 12.54 mls of buffer.

Calibration of the column with molecular weight standards implies a molecular weight for this protein of approximately 200 kDa. The expected molecular weight of the monomer is 28.08 kDa. This fusion protein is therefore also oligomeric in structure when purified from the cytosol of E. coli, without any steps being taken to refold it.

To verify that the fusion protein was indeed oligomeric, rather than that its behaviour on gel filtration was aberrant, the purified protein was denatured in 6M guanidinium chloride and 20 mM DTT (for 2 hours 30 minutes at room temperature) and the gel filtration repeated under denaturing conditions, (that is in the presence of 6M guanidinium chloride and 20 mM DTT).

Under these circumstance, the protein eluted in a volume of 12.5 mls, consistent with a molecular weight of approximately 220 kDa. The fusion protein is thus not denatured under these conditions: the protein is still oligomeric.

Complete denaturation of this protein was obtained after treatment with guanidine HCl for 16 hours at 4° C., in contrast to the Example 3 above, where heating to 75-80° C. was required to obtain complete denaturation.

Activity of the DsbA-C4bp Fusion Protein

To test the activity, of DsbA-C4bp, an insulin assay was conducted. In the presence of DTT, active DsbA catalyses the reduction of insulin's disulphide bonds which enables the separation of the two chains, and thus provokes the precipitation of the free insulin B chain. A turbidimetric assay is thus used to detect the reduction of the disulphide bonds of insulin. (Holmgren A (1979) Thioredoxin catalyses the reduction of insulin disulfides by dithiothreitol and dihydrolipoamide. J. Biol. Chem. 254, 9627).

The final reaction mixture contains 0.14 mM freshly prepared insulin, 0.1 M potassium phosphate pH 7.0, 2 mM EDTA, and 0.67 mM DTT, and 100 μg of DsbA or 100 μg of DsbA-C4bp fusion protein in a final volume of 1.2 ml.

The reaction was initiated by addition of 8 μl of 0.1 M DTT and monitored by measuring the increase of turbidity at 650 nm every 5 minutes up to 60 minutes. Each sample was gently mixed 3-4 times prior to measuring the absorbance at 650 nm. The instrument blank of the reaction contained 0.1 M phosphate buffer pH 7.0, and 2 mM EDTA.

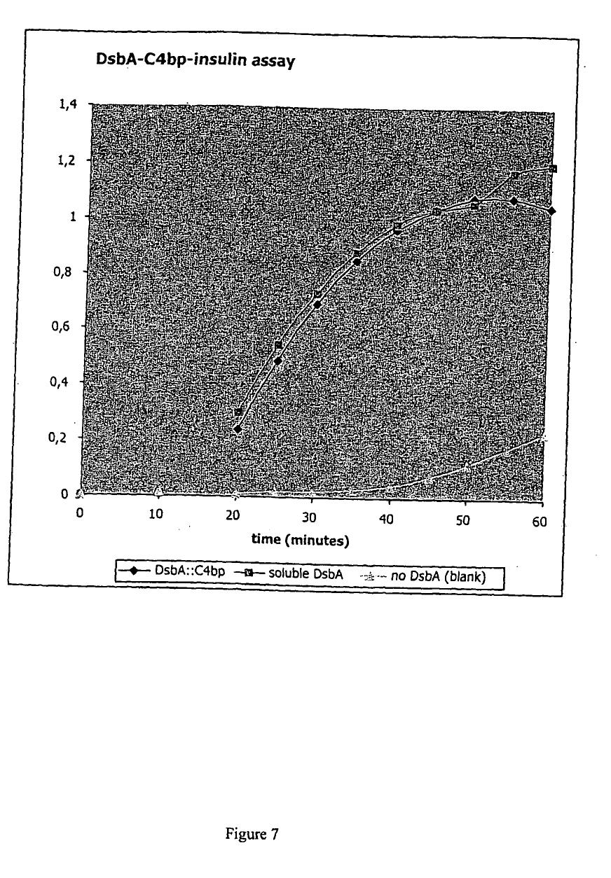

The results are shown in FIG. 7, and demonstrate that the DsbA present in the DsbA-C4bp fusion protein is still active, and the activity is directly comparable to the activity of soluble DsbA.

### EXAMPLE 6

### Analysis of C4bp Fusion Proteins Under Non-reducing Conditions

The analysis of the db-C4bp fusion protein by polyacrylamide gel electrophoresis under denaturing but non-reducing conditions was conducted to determine the presence or absence of disulphide bonds between the monomers of the oligomer.

Aliquots (12μl) of db-C4bp (312 μg/ml) were mixed with Laemmli buffer (Tris HCl 1.5 M pH 6.8, SDS 2%, glycerol 15%, 0.02% Bromophenol blue) with or without β-mercaptoethanol. These samples were boiled at 90° C. for 5 min and analysed by electrophoresis through a 18% sodium dodecyl sulfate polyacrylamide gel, also lacking β-mercaptoethanol.

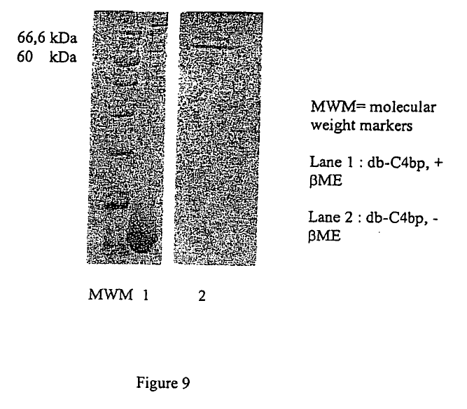

The result was that, in the absence of β-mercaptoethanol, the db-C4bp fusion protein migrated as an oligomer (in the top of the gel, FIG. 9 right side) showing that disulphide bonds exist between the monomers. In contrast, the addition of β-mercaptoethanol resulted in the migration of the db-C4bp protein at its monomer molecular weight, as shown in previous figures and on the left of FIG. 9 (showing reduced samples of db-C4bp during purification).

1. A method for obtaining a recombinant fusion protein comprising a scaffold of a C-terminal core protein of C4bp alpha chain, said recombinant fusion protein being capable of forming multimers in soluble form in a prokaryotic host cell, the method including the steps of
(i) providing a prokaryotic host cell carrying a nucleic acid encoding said recombinant protein operably linked to a promoter functional in said prokaryotic cell;
(ii) culturing the host cell under conditions wherein said recombinant protein is expressed; and
(iii) recovering the recombinant protein wherein said protein is recovered in multimeric form without performing a scaffold refolding step.

2. A method according to claim 1 wherein the recombinant protein is present at least at a concentration of at least 2 mg/l of cell culture.

3. A method according to claim 1 wherein the host prokaryotic cell is E. coli.

4. A method according to claim 3 wherein E. coli is selected from strain C41(DE3)[B96070444], C43(DE3)[B96070445] or CO214(DE3)[NCIMB40884], or other strains resistant to the toxicity of overexpressed recombinant proteins.

5. A method according to claim 1
wherein the recombinant protein comprises the C4bp core protein fused to a heterologous polypeptide.

6. A method according to claim 1 wherein said heterologous polypeptide is a TNF receptor protein.

7. A method according to claim 1 wherein said heterologous polypeptide is a BAFF-binding portion of BAFF-R.

8. A method according to claim 1 wherein said heterologous polypeptide is a thrombopoeitin agonist peptide IEGPTLRQWLAARA or somatostatin.

9. An isolated nucleic acid comprising a sequence which encodes a fusion protein of a C-terminal core protein of C4bp alpha chain and BAFF-R.

10. An isolated nucleic acid comprising a sequence which encodes a fusion protein of a C-terminal core protein of C4bp alpha chain and a thrombopoetin agonist peptide IEGPTLRQWLAARA or somatostatin.

11. A prokaryotic expression vector comprising a nucleic acid sequence encoding a fusion protein of a C-terminal core protein of C4bp alpha chain and a heterologous polypeptide operably linked to a promoter functional in prokaryotic cells.

12. A bacterial host cell transformed with the expression vector of claim 11.

13. A protein comprising a C-terminal core protein of C4bp alpha chain fused to BAFF-R.

14. A protein comprising a C-terminal core protein of C4bp alpha chain fused to a thrombopoeitin agonist peptide IEGPTLRQWLAARA.

15. A method according to claim 1 which further comprises formulating said recombinant protein into a composition comprising a pharmaceutically acceptable carrier or diluent.

16. A method for treating a condition in a patient, the
condition being associated with raised serum levels of BAFF, said method comprising the steps of administering to a patient a therapeutically effective amount of the protein of claim 14.

17. A method according to claim 16 wherein the condition is systemic lupus erythematosis.

18. A eukaryotic expression vector comprising a nucleic acid sequence encoding the protein of claim 13 operably linked to a promoter functional in eukaryotic cells.

19. A eukaryotic host cell transformed with the vector of claim 18.

20. Use of the expression vector of claim 18 in a method of treatment of the human or animal body.

21. A eukaryotic expression vector comprising a nucleic acid sequence encoding a recombinant fusion protein comprising a scaffold of a C-terminal core protein of C4bp alpha chain for the use in the treatment of the human or animal body.


SEQUENCE LISTING (29 sequences)

SEQ ID NO: 1 - protein, 57 aa, Homo sapiens
ETPEGCEQVLTGKRLMQCLPNPEDVKMALEVYKLSLEIEQLELQRDSARQSTLDKEL

SEQ ID NO: 2 - protein, 57 aa, Oryctolagus cuniculus
EVPEGCEQVQAGRRLMQCLADPYEVKMALEVYKLSLEIELLELQRDKARKSSVLRQL

SEQ ID NO: 3 - protein, 55 aa, Rattus sp.
EVPKDCEHVFAGKKLMQCLPNSNDVKMALEVYKLTLEIKQLQLQIDKAKHVDREL

SEQ ID NO: 4 - protein, 54 aa, Mus sp.
EASEDLKPALTGNKTMQYVPNSHDVKMALEIYKLTLEVELLQLQIQKEKHTEAH

SEQ ID NO: 5 - protein, 67 aa, Bos sp.
EYPEGCEQVVTGRKLLQCLSRPEEVKLALEVYKLSLEIEILQTNKLKKEAFLLREREKNVTCDFNPE

SEQ ID NO: 6 - protein, 57 aa, Sus scrofa
EYPEDCEQVHEGKKLMECLPTLEEIKLALALYKLSLETNLLELQIDKEKKAKAKYST

SEQ ID NO: 7 - protein, 56 aa, Cavia porcellus
EVPEECKQVAAGRKLLECLPNPSDVKMALEVYKLSLEIEQLEKEKYVKIQEKFSKE

SEQ ID NO: 8 - protein, 59 aa, Mus sp.
EVLEDCRIVSRGAQLLHCLSSPEDVHRALKVYKLFLEIERLEHQKEKWIQLHRKPQSMK

SEQ ID NO: 9 - protein, 52 aa, Artificial Sequence
  Description of Artificial Sequence Variant of the C4bp core protein
CEQVLTGKRLMQCLPNPEDVKMALEVYKLSLEIEQLELQRDSARQSTLDKEL

SEQ ID NO: 10 - protein, 57 aa, Artificial Sequence
  Description of Artificial Sequence Variant of the C4bp core protein
ETPEGCEQVLTGKRLMQCLPNPEDVKMALEVYKLSLEIKQLELQRDSARQSTLDKEL

SEQ ID NO: 11 - protein, 52 aa, Artificial Sequence
  Description of Artificial Sequence Variant of the C4bp core protein
CEQVLTGKRLMQCLPNPEDVKMALEVYKLSLEIKQLELQRDSARQSTLDKEL

SEQ ID NO: 12 - protein, 57 aa, Artificial Sequence
  Description of Artificial Sequence Variant of the C4bp core protein
ETPEGCEQVLTGKRLMQCLPNPEDVKMALEIYKLSLEIEQLELQRDSARQSTLDKEL

SEQ ID NO: 13 - protein, 57 aa, Artificial Sequence
  Description of Artificial Sequence Variant of the C4bp core protein
ETPEGCEQVLTGKRLMQCLPNPEDVKMALEIYKLSLEIKQLELQRDSARQSTLDKEL

SEQ ID NO: 14 - protein, 50 aa, Artificial Sequence
  Description of Artificial Sequence Variant of the C4bp core protein
EGCEQALTGKRLMQCLPNPEDVKMALEIYKLSLEIKQLELQRDSARQSTL

SEQ ID NO: 15 - protein, 57 aa, Artificial Sequence
  Description of Artificial Sequence Variant of the C4bp core protein
ETPEGSEQVLTGKRLMQSLPNPEDVKMALEVYKLSLEIKQLELQRDSARQSTLDKEL

SEQ ID NO: 16 - protein, 52 aa, Artificial Sequence
  Description of Artificial Sequence Variant of the C4bp core protein
EGSEQALTGKRLMQSLPNPEDVKMALEIYKLSLEIEQLELQRDSARQSTLDK

SEQ ID NO: 17 - protein, 5 aa, Artificial Sequence
  Description of Artificial Sequence Flexible linker
GGGGS

SEQ ID NO: 18 - protein, 10 aa, Artificial Sequence
  Description of Artificial Sequence Flexible linker
GGGGSGGGGS

SEQ ID NO: 19 - protein, 15 aa, Artificial Sequence
  Description of Artificial Sequence Flexible linker
GGGGSGGGGSGGGGS

SEQ ID NO: 20 - protein, 20 aa, Artificial Sequence
  Description of Artificial Sequence Flexible linker
GGGGSGGGGSGGGGSGGGGS

SEQ ID NO: 21 - protein, 14 aa, Artificial Sequence
  Description of Artificial Sequence Thrombopoeitin agonist peptide
IEGPTLRQWLAARA

SEQ ID NO: 22 - protein, 9 aa, Artificial Sequence
  Description of Artificial Sequence Downstream box peptide sequence
MASMNHKGS

SEQ ID NO: 23 - DNA, 31 bp, Artificial Sequence
  Description of Artificial Sequence Primer
cccgcggatccgagacccccgaaggctgtga

SEQ ID NO: 24 - DNA, 37 bp, Artificial Sequence
  Description of Artificial Sequence Primer
ccccggaattcttattatagttctttatccaaagtgg

SEQ ID NO: 25 - DNA, 54 bp, Artificial Sequence
  Description of Artificial Sequence Sequence encoding a 6xHistidine tag
catatgcggggttctcatcatcatcatcatcatggtctggttccgcgtggatcc

SEQ ID NO: 26 - protein, 74 aa, Artificial Sequence
  Description of Artificial Sequence Amino acid sequence  produced by plasmid pAVD 93
MRGSHHHHHHGLVPRGSETPEGCEQVLTGKRLMQCLPNPEDVKMALEVYKLSLEIEQLELQRDSARQSTLDKEL

SEQ ID NO: 27 - DNA, 38 bp, Artificial Sequence
  Description of Artificial Sequence Primer
ggggcccccatatggcgcagtatgaagatggtaaacag

SEQ ID NO: 28 - DNA, 48 bp, Artificial Sequence
  Description of Artificial Sequence Primer
ggggaattcttaggatccagaaccttttttctcggacagatatttcac

SEQ ID NO: 29 - DNA, 303 bp, Artificial Sequence
  Description of Artificial Sequence Promoter and C4bp coding region in pAVD77
gatctcgatcccgcgaaattaatacgactcactatagggagaccacaacggtttccctctagaaataattttgtttaactttaagaaggagatatacatatggctagcatgaatcacaaaggatccgagacccccgaaggctgtgaacaagtgctcacaggcaaaagactcatgcagtgtctcccaaacccagaggatgtgaaaatggccctggaggtatataagctgtctctggaaattgaacaactggaactacagagagacagcgcaagacaatccactttggataaagaactataataa